(19) United States
(12) Patent Application Publication
(10) Pub. No.: US 2024/0148434 A1
(43) Pub. Date: May 9, 2024

(54) ABLATION CATHETERS HAVING FLUSHING CAPABILITY

(71) Applicant: Oscor Inc., Palm Harbor, FL (US)

(72) Inventors: Noel Perez, Palm Harbor, FL (US); Thomas P. Osypka, Palm Harbor, FL (US)

(21) Appl. No.: 18/406,532

(22) Filed: Jan. 8, 2024

Related U.S. Application Data
(63) Continuation-in-part of application No. 17/482,212, filed on Sep. 22, 2021.
(60) Provisional application No. 63/082,398, filed on Sep. 23, 2020.

(51) Int. Cl. A61B 18/14

(52) U.S. Cl. CPC A61B 18/1492; A61B 2018/00267

(57) ABSTRACT

A catheter assembly comprises a catheter having an axial lumen that houses electrical wires. A plurality of splines, each supporting at least one electrode, are connected to the wires and a distal cap. The splines have a basket shape in a deployed position and a collapsed shape when received inside the catheter axial lumen. A flexible tube extending outwardly from the catheter lumen has a tube lumen of a first, inner diameter and a distal tube end that is spaced proximally from the distal cap. A guide shaft extending proximally from the distal cap has a second, outer diameter that is less than the first diameter of the tube lumen. With the guide shaft received in the tube lumen, an annulus is formed by the tube lumen surrounding the guide shaft. With the splines in the deployed position, the annulus permits flushing of the area adjacent to the deployed splines.

### CROSS-REFERENCE TO RELATED APPLICATIONS

This application is a continuation-in-part application of U.S. application Ser. No. 17/482,212, filed on Sep. 22, 2021, now abandoned, which claims priority to U.S. provisional application Ser. No. 63/082,398, filed Sep. 23, 2020.

### BACKGROUND OF THE INVENTION

### 1. Field of the Invention

This disclosure relates to ablation catheters and systems, e.g., to ablation catheters for performing renal denervation procedures through the renal artery of a patient.

### 2. Prior Art

Renal denervation (RDN) is a procedure performed by interventional radiologists for the purpose of lowering the blood pressure of a patient. Renal denervation is a minimally invasive, endovascular catheter-based procedure using radiofrequency (RF) ablation aimed at treating resistant hypertension.

By applying RF energy to the renal arteries, the nerves in the vascular wall (adventitia layer) can be denervated. This causes reduction of renal sympathetic afferent and efferent activity which in turn can decrease blood pressure. Early data from international clinical trials demonstrates average blood pressure reduction of approximately 30 mm Hg at three-year follow-ups in patients with treatment-resistant hypertension.

A common way to perform renal ablation is to ablate the renal artery by either heating tissue through radiofrequency or microwave ablation, irrigated heat ablation, and/or cryogenic ablation. It is believed that renal denervation works because it reduces the over-activity of the sympathetic nerve. Ablation of the renal artery is commonly performed by gaining access through the femoral vein. However, in certain cases, this can cause substantial bleeding. Other options include access through the radial artery. But this method limits the use of catheter systems of SF (French size) or smaller.

Current ablation catheters that are available to the market include: 1) single polar catheters offered by Medtronic of 45 710 Medtronic Parkway, Minneapolis, Minn., 55432-5604, which take substantial time to perform effective ablation of the renal artery; 2) cage form catheters offered by St. Jude Medical of One St. Jude Medical Drive, St. Paul, Minn., 55117-9983, which have several electrodes configured in a cage form; and 3) multiple ablation electrodes configured on an inflatable balloon, like those offered by Boston Scientific of One Boston Scientific Place, Natick, Mass., 01760-1537.

All unipolar renal denervation catheters have a major disadvantage. Unipolar catheters requires the use of a monopolar-based patient grounding pad therefore the ablation energy applied through the positive electrode or electrodes flows throughout the body into the grounding pad. Current will flow between the unipolar electrode or electrodes from inside the renal artery to the grounding pad typically located on the patient's back. Current flow between the two electrodes will increase the temperature of the tissue and organs located between the two electrodes and the physician cannot control the tissues or organs impacted by the temperature increase. The unipolar catheter also requires higher treatment time to achieve the RF ablation due to the grounding pad location. Additionally, unipolar RDN catheters require the use of cooling and irrigation systems to prevent overheating and damaging the renal artery walls and the circulating blood during the RF ablation procedure. The disadvantage in current bipolar balloon-based ablation catheters is that the blood flow through the renal artery is blocked while the balloon is inflated increasing the risk to the patient.

Another shortcoming of current renal denervation systems is that even though the physician can observe the positioning of the ablation catheter in the renal artery through contrast media supported X-ray, the physician does not know the location of the sympathetic nerves of the renal artery and, therefore, does not know the correct and ideal position of the catheter to be placed to make the actual ablation and treatment time as short and efficient as possible. Physicians are essentially performing this procedure blind with presently available devices, and the only available approach to the physician is to perform ablation to all nerves surrounding the renal artery in several places along the renal artery. Even though it is well known that the over-activity of the renal sympathetic nerves are responsible for higher blood pressure in a patient, the actual place or location of the nerve path with the over-activity is not measured to be able to identify the correct location to perform a targeted ablation of the nerve.

Nonetheless, conventional ablation methods and systems have generally been considered satisfactory for their intended purpose. However, there is still a need in the art for improved catheter-based ablation systems. There also remains a need in the art for a system that is easy to make and use. The present disclosure provides a solution for these problems.

### SUMMARY OF THE INVENTION

A catheter assembly can include an elongated catheter having opposed proximal and distal end portions and an axial lumen extending therethrough, the axial lumen being configured to accommodate electrical wires. The assembly can include a distal electrode array configured to have a basket shape in a deployed position and configured to collapse in a collapsed position. The distal electrode array is configured to extend from the distal end portion of the elongated catheter in the deployed position and to be selectively contained within the axial lumen or other sheath in the collapsed position.

The assembly can include a plurality of electrical wires electrically connected to the distal electrode array and disposed through the axial lumen, a proximal hub connected to the elongated catheter at the proximal end, and an electrical plug connected to the proximal hub and electrically connected to the plurality of electrical wires. The assembly can include a flexible tube disposed in the axial lumen of the elongated catheter, and a tubing having a side port with a multiway stop cock side port such that the catheter assembly can be flushed. In certain embodiments, the assembly can include an introducer for loading the elongated catheter and/or distal electrode array into a deflectable guiding sheath for delivery of the elongated catheter and/or distal electrode array through femoral access into the renal nerves.

The distal electrode array can include two or more (e.g., four) splines configured to be collapsible and adjustable. The splines can be made of pre-shaped shape-memory material wire. In certain embodiments, the shape-memory material wire is covered with a thermoplastic polyurethane (TPU). In certain embodiments, the shape-memory material wires can be terminated with a distal cap.

The distal electrode array can include a plurality of ablation electrodes. For example, a four-spline configuration can include sixteen ablation electrodes such that four ablation electrodes can be attached to a top of each of the four splines. Each ablation electrode can be electrically attached to an electrical wire of the plurality of electrical wires. The electrodes can have a thermoplastic polyurethane (TPU) cover with a cutout window to expose the electrodes surface to come in contact with the ablated surface.

The flexible tube extends outwardly from the catheter lumen and has a tube lumen of a first, inner diameter. A distal tube end of the flexible tube is spaced proximally from the distal cap. A guide shaft extending proximally from the distal cap has a second, outer diameter that is less than the first diameter of the tube lumen. With the guide shaft received in the tube lumen, an annulus is formed by the tube lumen surrounding the guide shaft. With the splines in the deployed position, the annulus permits flushing of the area adjacent to the deployed splines.

The distal electrode array can include two or more orientation electrodes, at least one disposed on two or more of the splines, and configured for orientation under fluoroscopy. In certain embodiments, the orientation electrodes may not be connected to the electrical wires. The axial lumen can be terminated at the distal end with adhesive along with a cross-shaped fixture to hold the plurality of splines in place and allow the splines to collapse and expand.

In certain embodiments, the assembly can include a switching module configured to connect to the electrical plug and the electrodes, and to connect to a recorder module configured to receive signals from the electrode array and an ablation energy generator to provide selection between electrical mapping and ablation energy generation. In certain embodiments, the ablation electrodes and/or the distal cap and/or the orientation electrodes are made of platinum iridium (e.g., cylindrical platinum iridium electrodes).

In accordance with at least one aspect of this disclosure, an ablation catheter system can include: a recorder module configured to receive sympathetic signals from the circumference of the artery to allow the location of the nerve path with the renal sympathetic over-activity, an ablation energy generator configured to output power to the ablation electrodes to cause tissue ablation, and a switching module configured to switch between the recorder module and an ablation energy generator to switch between electrical mapping and ablation of the selected zone with the renal sympathetic over-activity utilizing the same electrode array.

In accordance with at least one aspect of this disclosure, an electrode array for renal ablation can include a basket shape in a deployed position and configured to collapse in a collapsed position, the electrode array configured to extend from a distal end portion of an elongated catheter in the deployed position and to be selectively contained within the axial lumen or other sheath in the collapsed position. The electrode array can include two or more splines configured to be collapsible and adjustable.

The splines can be made of pre-shaped shape-memory material wire. The distal electrode array can include a plurality of ablation electrodes (e.g., each spline having multiple electrodes). In certain embodiments, the plurality of electrodes can be sixteen ablation electrodes, with four ablation electrodes attached to a top of each of the splines. Each ablation electrode can be electrically attached to an electrical wire of the plurality of electrical wires. The distal electrode array can include two or more orientation electrodes, at least one disposed on two or more of the splines, and configured for orientation under fluoroscopy. The orientation electrodes may not be connected to the electrical wires. The electrode array can be or include any suitable embodiment of an electrode array disclosed herein, e.g., as described above.

These and other features of the embodiments of the subject disclosure will become more readily apparent to those skilled in the art from the following detailed description taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, embodiments thereof will be described in detail herein below with reference to certain figures, wherein:

FIG. 1 is a schematic elevation view of an embodiment of a catheter assembly in accordance with this disclosure.

FIG. 2A is a schematic elevation view of an end effector portion of the catheter assembly as shown in FIG. 1.

FIG. 2B is a schematic elevation view of a portion of an electrode assembly of the embodiment of FIG. 2A.

FIG. 2C is a cross-sectional view of the elongate catheter of the embodiment of FIG. 2A.

FIG. 3 is a schematic diagram of an embodiment of a switching module operatively connected to an electrical mapping module and an RF generator module, and the catheter, e.g., as shown in FIG. 1.

FIG. 4 is a schematic diagram of the embodiment of FIGS. 1 and 2 being inserted into a renal artery.

### DETAILED DESCRIPTION OF THE PREFERRED EMBODIMENTS

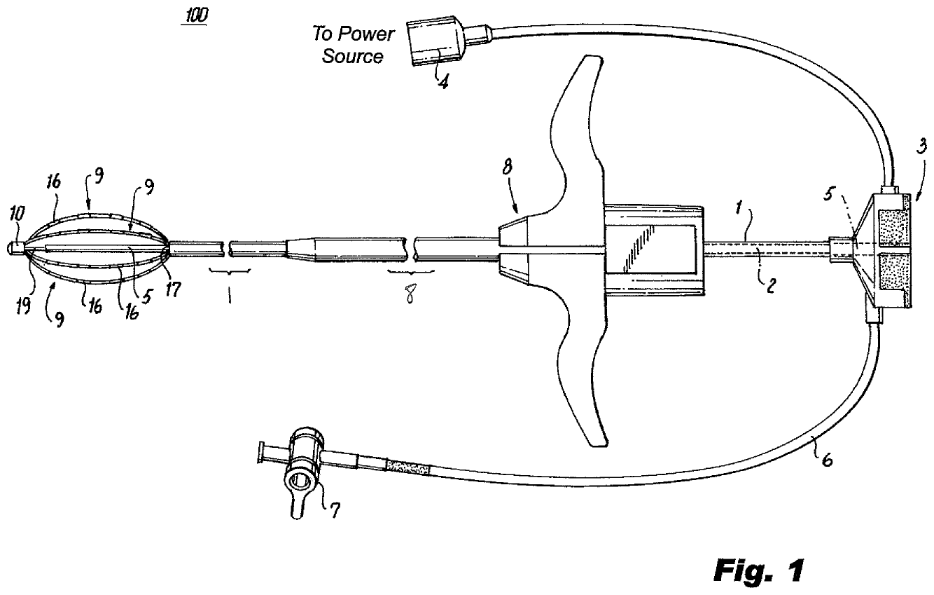

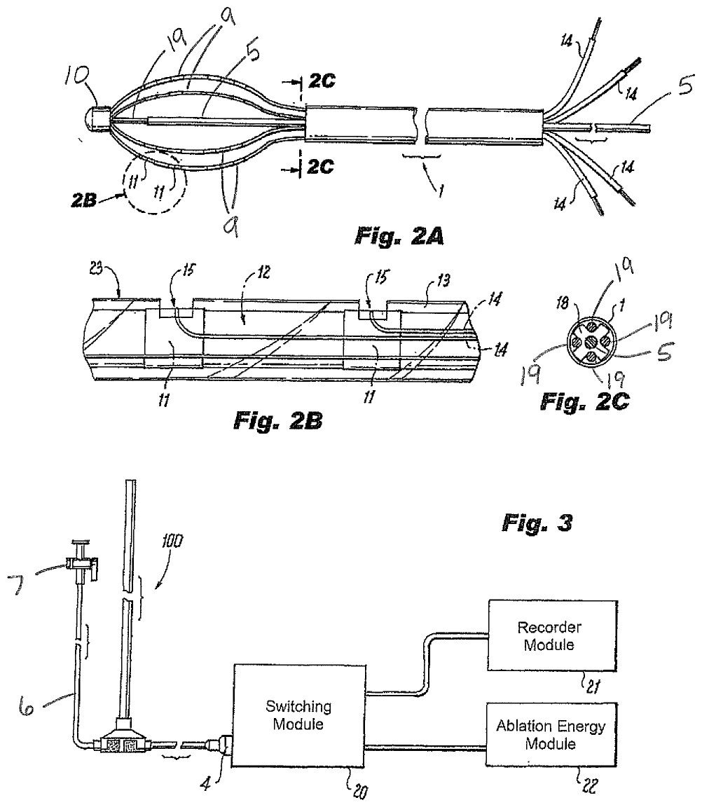

Reference will now be made to the drawings wherein like reference numerals identify similar structural features or aspects of the subject disclosure. For purposes of explanation and illustration, and not limitation, an illustrative view of an embodiment of an ablation catheter in accordance with the disclosure is shown in FIG. 1 and is designated generally by reference character 100. Other embodiments and/or aspects of this disclosure are shown in FIGS. 2A-4. Certain embodiments disclosed herein can be used for ablating the interior walls of the renal artery to reduce renal sympathetic afferent and efferent activity, among other things.

In accordance with at least one aspect of this disclosure, referring to FIGS. 1, 2A, 2B, and 2C, a catheter assembly 100 can include an elongated catheter 1 having opposed proximal and distal end portions and having an axial lumen 2 extending therethrough. The axial lumen 2 can be configured to accommodate electrical wires 14 (e.g., insulated). The assembly 100 can include a distal electrode array 101 configured to have a basket shape (e.g., a skeleton approximating a balloon shape as shown or any other suitable shape) in a deployed position (e.g., as shown) and configured to collapse in a collapsed position (e.g., to be linear to fit within a distal end of the axial lumen 2 or other sheath). The distal electrode array 101 can be configured to extend from the distal end portion of the elongated catheter 1 in the deployed position, e.g., as shown, and to be selectively contained within the axial lumen 2 or other sheath in the collapsed position, for example. Any other suitable relative relationship to the elongated catheter 1 is contemplated herein.

The assembly 100 can include a plurality of electrical wires 14 electrically connected to the distal electrode array 101 and disposed through the axial lumen 2, a proximal hub 3 connected to the elongated catheter 1 at the proximal end, and an electrical plug 4 connected to the proximal hub 3 and electrically connected to the plurality of electrical wires 14. The assembly 100 can include a flexible tube 5 (e.g., made of polyimide or other suitable material) disposed in the axial lumen 2 of the elongated catheter 1. The assembly 100 can include tubing 6 having a side port with a multiway (e.g., two or three way) stop cock side port 7 such that the catheter assembly can be flushed.

In certain embodiments, the assembly 100 can include an introducer 8 for loading the elongated catheter 1 and/or distal electrode array 101 into a deflectable guiding sheath for delivery of the elongated catheter 1 and/or distal electrode array 101 through femoral access into the renal nerves. Any suitable introducer 8 is contemplated herein (e.g., an Adelante® Peel-Away Introducer made by Oscor, Inc. Palm Harbor Fl, 34683), e.g., for loading the basket catheter into a Destinom deflectable guiding sheath made by Oscor, Inc. Palm Harbor Fl, 34683, or any other suitable guiding sheath is contemplated herein.

The distal electrode array 101 can include two or more splines 9 (e.g., four as shown) configured to be collapsible and adjustable. Any other suitable number of splines (e.g., two to six) that form a basket shape is contemplated herein. The splines 9 can extend radially outward, each in a bow shape, e.g., as shown, to form the basket shape.

The splines 9 can be made of pre-shaped shape-memory (e.g., Nitinol) material wire 12. In certain embodiments, the shape-memory material wire 12 can be covered with a thermoplastic polyurethane (TPU) (e.g., Pellethane®) 13 or any other suitable electrical insulating material. In certain embodiments, the shape-memory material wires 12 can be terminated with a cylindrical cap 10 or any other suitable tip (e.g., a smooth surface).

The distal electrode array 101 can include a plurality of ablation electrodes 11 (e.g., a plurality of electrodes on each spline 9). For example, for a four-spline configuration, the distal electrode array 101 can include sixteen ablation electrodes 11, four ablation electrodes 11 attached (e.g., glued) to a top of each of the four splines 9, e.g., as shown. Any other suitable number of electrodes 11 is contemplated herein.

Each ablation electrode 11 can be electrically attached (e.g., laser welded) to an electrical wire 14 (e.g., insulated wires 15) of the plurality of electrical wires 14. Each pair of electrodes 11 can have a thermoplastic polyurethane (TPU) cover therebetween. In certain embodiments, each electrode 11 can have a TPU cover 23 with a cutout window to expose the electrodes surface to come in contact with the ablated surface, e.g., as shown in FIG. 2B. As shown, a first pair of electrodes 11 on each spline can be positioned distally of an apex of the curvature of each spline 9, and a second pair of electrodes 11 can be placed proximally of the apex of each spline 9. Any other suitable arrangement is contemplated herein. Any other suitable material other than TPU is contemplated herein.

The distal electrode array 101 can include two or more orientation electrodes 16, at least one disposed on two or more of the splines 9, e.g., as shown, and configured for orientation under fluoroscopy for example. The orientation electrodes 16 may not be connected to the electrical wires 14. The orientation electrodes 16 can be placed distally of the ablation electrodes 11 on each spline 9. Any other suitable position is contemplated herein.

The axial lumen 2 can be terminated at its distal end with adhesive 17 along with a cross-shaped fixture 18 to hold the plurality of splines 9 in place and allow the splines 9 to collapse and expand. The flexible tube 5 extends through the axial lumen of the catheter 1 and through a center opening in the cross-shaped fixture 18 at the distal open end of the elongated catheter. The flexible tube 5 has a tube lumen of a first, inner diameter, but is devoid of any perforations or openings through its sidewall. A distal end of the flexible tube 5 extends distally outwardly from the cross-shaped fixture 18 but is spaced proximally with respect to the distal cap 10.

A guide shaft 19 extends proximally from the distal cap 10. The guide shaft 19 is preferably a solid member that is devoid of perforations. A proximal portion of the guide shaft having a second, outer diameter that is less than the first, inner diameter of the flexible tube lumen is received in a moveable relationship inside the flexible tube lumen. With the guide shaft of the second, lesser outer diameter received in the tube lumen of the first, greater inner diameter, an annulus is provided that is sufficient to permit flushing the area adjacent to the splines 9 when the splines are in the deployed configuration shown in FIGS. 1, 2A and 4. The flushing fluid is introduced into the tubing 6 shown in FIGS. 1 and 3 from the side port 7 to flow through the axial lumen 2 of the flexible tube 5 and out through the distal end of the tube 5 to flush the deployed splines 9. The guide shaft 19 can be made of any suitable rigid or semi-rigid material (e.g., stainless steel), and is attached to the distal cap 10 (e.g., bonded) in any suitable manner.

The term “annulus” is defined as the annular void formed by the inner lumen of the first diameter of the flexible tube surrounding the outer surface of the guide shaft of the second diameter with the first diameter being greater than the second diameter.

In certain embodiments, referring to FIG. 3, the assembly 100 can include a switching module 20 configured to connect to the electrical plug 4 and the electrodes 11, 16, and to connect to a recorder module 21 configured to receive signals from the electrode array 101 and an ablation energy generator 22 (e.g., an RF generator) to provide selection between electrical mapping and ablation energy generation. The switching module 20 can include any suitable hardware or software module configured to allow switching between mapping and ablation (e.g., due to a manual input to switch, or in accordance with any suitable schedule or logic).

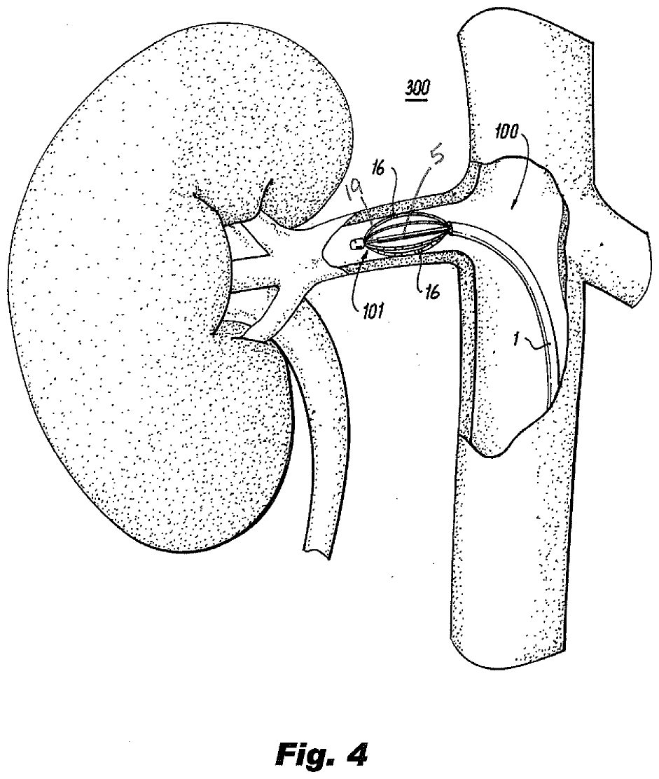

FIG. 4 shows a schematic diagram of the embodiment of FIGS. 1 and 2 being inserted into a renal artery. As shown, the elongate catheter 1 can be compliant and steerable (e.g., configured to bend or make angled turns (e.g., 90 degrees). Any suitable arrangement for steering the device into a suitable anatomical location is contemplated herein.

In certain embodiments, the ablation electrodes, the distal cap, and the orientation electrodes are made of platinum iridium (e.g., cylindrical platinum iridium electrodes). Any other suitable material and shape is contemplated herein.

In accordance with at least one aspect of this disclosure, an ablation catheter system (e.g., system 300 as shown in FIG. 3) can include a recorder module 21 configured to receive sympathetic signals from the circumference of the artery to allow the location of the nerve path with the renal sympathetic over-activity to be recorded. The system 300 can include an ablation energy generator 22 configured to output power to the ablation electrodes 11 to cause tissue ablation. The system 300 can include a switching module 20 configured to switch between the recorder module 21 and an ablation energy generator 22 to switch between electrical mapping and ablation of the selected zone with the renal sympathetic over-activity utilizing the same electrode array 101.

In accordance with at least one aspect of this disclosure, an electrode array 101 for renal ablation can include a basket shape in a deployed position and configured to collapse in a collapsed position, the electrode array 101 configured to extend from a distal end portion of an elongated catheter in the deployed position and to be selectively contained within the axial lumen or other sheath in the collapsed position. The electrode array 101 can include two or more (e.g., four) splines 9 configured to be collapsible and adjustable.

The splines 9 can be made of pre-shaped shape-memory material wire. The distal electrode array 101 can include a plurality of electrodes on each spline. For example, certain embodiments can include sixteen ablation electrodes with four ablation electrodes attached to a top of each of the splines 9. Each ablation electrode can be electrically attached to an electrical wire 14 of the plurality of electrical wires 14. The distal electrode array 101 can include two or more orientation electrodes 16, at least one disposed on two or more of the splines 9, and configured for orientation under fluoroscopy. The orientation electrodes 16 may not be connected to the electrical wires 14 that are connected to the ablation electrodes 11, for example. The electrode array 101 can be or include any suitable embodiment of an electrode array 101 disclosed herein, e.g., as described above.

In accordance with at least one aspect of this disclosure, certain embodiments of a system (e.g., system 100) for use in a renal denervation procedure can include a catheter having proximal and distal end portions, a series of splines with pre-shaped geometry with memory to collapse and return to a basket shape, the electrode array being assembled over the basket splines to measure the renal sympathetic activity around the renal artery with the same electrode array on the distal end portion of the catheter for delivering energy to ablate the renal tissue with nerves surrounding the renal artery. Each pair of electrodes can have a Pellethane® cover between them to reduce current density at the edges which protects the artery wall from ablation and channels the current flow through the tissue beyond walls for an effective ablation of the target area.

The system can further include a catheter handle (e.g., proximal hub 3) at the proximal end portion of the catheter wherein the handle is connectable to a multiplexer or switch box that is configured to either perform mapping of renal sympathetic nerve activity or provide energy to a selectable pair of electrodes for ablation of the renal artery. The catheter handle can include an actuation portion for facilitating bidirectional steering of the distal end portion of the catheter within the renal artery. An overall diameter of the catheter can be less than about 6 F, for example. Any suitable handle structure, e.g., as appreciated by those having ordinary skill in the art, for steering the catheter is contemplated herein.

The distal end portion of the catheter can have a generally basket shaped configuration. In some embodiments, the distal end portion of the catheter can have two to six splines forming the basket shape, for example. Any suitable number of splines is contemplated herein. The system can further include a radio frequency generator operatively connected to the catheter handle to provide energy to the plurality of electrodes for ablation of the renal artery.

In at least one aspect of this disclosure, a method can include inserting the catheter into a renal artery and sensing a condition associated with a nerve of a renal artery using the electrodes on the catheter. The method can further include determining whether to ablate tissue based on the sensed condition of the nerves. The method can further include ablating tissue if the nerves are sensed to be over-active. The method can include any other suitable method and portion thereof.

In at least one aspect of this disclosure, a catheter can include a catheter body defining a distal end portion and a proximal end portion, and an electrode array 101 for sensing a renal sympathetic nerve, the electrodes disposed on the distal end portion of the catheter body. The electrodes can be configured to sense the electro-chemical signals from the renal sympathetic nerves. The catheter can further include the electrode array 101 to be electrically connected to an electro-surgical energy source generator. In certain embodiments, a diameter of the catheter body can be less than about 6 F.

The catheter can further include a catheter handle at a proximal end portion of the catheter body wherein the handle is connectable to a generator that is configured to provide energy to any selectable pair of electrodes for ablation of a renal artery location. The catheter handle can include an actuation portion for steering the distal end portion of the catheter body within the renal artery. Any suitable handle and steering assembly is contemplated herein.

Embodiments can include a system for use in a renal denervation procedure having a catheter having proximal and distal end portions, a sensor configuration to monitor the condition of the nerves surrounding the renal artery, the sensor array operatively associated with the distal end portion of the catheter, and with the same sensor array on the distal end portion of the catheter for delivering energy to renal surrounding tissue. A catheter can include a catheter body defining a distal end portion and a proximal end portion, and a sensor array for sensing a renal sympathetic nerve activity. The sensor array can be configured to sense the electrical signals from the renal sympathetic nerves surrounding the renal artery and selectively perform renal ablation at the target area.

Those having ordinary skill in the art understand that any numerical values disclosed herein can be exact values or can be values within a range. Further, any terms of approximation (e.g., “about”, “approximately”, “around”) used in this disclosure can mean the stated value within a range. For example, in certain embodiments, the range can be within (plus or minus) 20%, or within 10%, or within 5%, or within 2%, or within any other suitable percentage or number as appreciated by those having ordinary skill in the art (e.g., for known tolerance limits or error ranges).

The embodiments of the present disclosure, as described above and shown in the drawings, provide for improvement in the art to which they pertain. While the subject disclosure includes reference to certain embodiments, those skilled in the art will readily appreciate that changes and/or modifications may be made thereto without departing from the spirit and scope of the subject disclosure.

What is claimed is:

1. A catheter assembly, comprising:
a) an elongated catheter comprising an inner surface defining an axial lumen extending from a catheter proximal open end to an opposed distal open end of the catheter;
b) a plurality of electrical wires extending from the catheter proximal open end, through the axial lumen and to the distal open end of the catheter;
c) a distal electrode array comprising a plurality of pre-shaped shape-memory splines connected to a respective one of the plurality of electrical wires, wherein at least one electrode of the distal electrode array is electrically connected to a respective one of the pre-shaped shape-memory splines;
d) a distal cap connected to a distal end of the plurality of pre-shaped shape-memory splines; and
e) a flexible tube extending through the axial lumen of the catheter, wherein the flexible tube has a tubular sidewall defining a tube lumen of a first, inner diameter, and a distal end of the flexible tube is spaced proximally with respect to the distal cap; and
f) a guide shaft having a second, outer diameter that is less than the first, inner diameter of the flexible tube lumen, wherein the guide shaft extends proximally from the distal cap and is received in the tube lumen of the flexible tube, and wherein with the guide shaft received in the tube lumen, an annulus is formed by the tube lumen of the first diameter surrounding the guide shaft of the second diameter,
h) wherein the plurality of pre-shaped shape-memory splines are configured to move between a collapsed position received inside the axial lumen of the catheter, and a basket-shaped, deployed position extending outwardly from the catheter distal open end, and with the plurality of pre-shaped shape-memory splines in the deployed position, the annulus between the outer surface of the guide shaft surrounded by the inner lumen of the flexible tube is sufficient to permit flushing the area adjacent to the deployed splines.

2. The catheter assembly of claim 1, wherein a proximal portion of the guide shaft is in a movable relationship inside the tube lumen of the flexible tube.

3. The catheter assembly of claim 1, further comprising a cross-shaped fixture located at the distal open end of the catheter, wherein there are four electrical wires and four pre-shaped shape memory splines with one of the four pre-shaped shape memory splines being connected to a respective one of the four electrical wires extending through a respective one of four openings formed by the cross-shaped fixture and the inner surface of the catheter.

4. The catheter assembly of claim 3, wherein the cross-shaped fixture has a center opening and the flexible tube extends through the center opening in the cross-shaped fixture at the distal open end of the elongated catheter.

5. The catheter assembly of claim 1, further comprising:
a) a proximal hub connected to the proximal catheter open end; and
b) an electrical plug electrically connected to the plurality of electrical wires at the proximal hub.

6. The catheter assembly of claim 5, further comprising a tubing extending from the proximal hub at the proximal catheter open end to a multiway stop cock.

7. The catheter assembly of claim 5, further comprising a switching module that is configured to selectively connect a recorder module and an ablation energy generator to the electrical plug, wherein the recorder module is configured to receive electrical mapping signals from the distal electrode array, and the ablation energy generator is configured to provide electrical ablation energy to the distal electrode array.

8. The catheter assembly of claim 1, wherein at least one of the pre-shaped shape-memory wires is covered with a thermoplastic polyurethane (TPU).

9. The catheter assembly of claim 1, wherein the at least one electrode of the distal electrode array comprises four electrodes that are attached to each of the plurality of pre-shaped shape-memory wires.

10. The catheter assembly of claim 1, wherein the at least one electrode of the distal electrode array electrically connected to the respective one of the plurality of pre-shaped shape-memory wires has a thermoplastic polyurethane (TPU) cover with a cutout window to expose a surface of the at least one electrode.

11. The catheter assembly of claim 1, wherein at least two orientation markers are disposed on a respective one of the plurality of pre-shaped shape-memory wires, and wherein the orientation markers are not electrically connected to the plurality of electrical wires.

12. The catheter assembly of claim 1, wherein the tubular sidewall of the flexible tube is devoid of any perforations or openings.

13. An ablation catheter system, comprising:
a) catheter assembly, comprising:
i) an elongated catheter comprising an inner surface defining an axial lumen extending from a catheter proximal open end to an opposed distal open end of the catheter;
ii) a plurality of electrical wires extending from the catheter proximal open end, through the axial lumen and to the distal open end of the catheter;
iii) a distal electrode array comprising a plurality of pre-shaped shape-memory splines connected to a respective one of the plurality of electrical wires, wherein at least one electrode of the distal electrode array is electrically connected to a respective one of the plurality of pre-shaped shape-memory splines;
iv) a distal cap connected to a distal end of the plurality of pre-shaped shape-memory splines;
v) a flexible tube extending through the axial lumen of the catheter, wherein the flexible tube has a tube lumen of a first, inner diameter, and a distal end of the flexible tube is spaced proximally with respect to the distal cap; and
vi) a guide shaft having a second, outer diameter that is less than the first, inner diameter of the flexible tube lumen, wherein the guide shaft extends proximally from the distal cap and is received in the tube lumen of the flexible tube, and wherein with the guide shaft received in the tube lumen, an annulus is formed by the tube lumen of the first diameter surrounding the guide shaft of the second diameter,
vii) wherein the plurality of pre-shaped shape-memory splines are configured to move between a collapsed position received inside the axial lumen of the catheter, and a basket-shaped, deployed position extending outwardly from the catheter distal open end, and with the plurality of pre-shaped shape-memory splines in the deployed position, the annulus between the outer surface of the guide shaft surrounded by the inner lumen of the flexible tube is sufficient to permit flushing the area adjacent to the deployed splines; and
b) a switching module that is configured to selectively connect a recorder module and an ablation energy generator to the catheter assembly, wherein the recorder module is configured to receive electrical mapping signals from the distal electrode array, and the ablation energy generator is configured to provide electrical ablation energy to the distal electrode array.

14. The ablation catheter system of claim 13, wherein a proximal portion of the guide shaft is in a movable relationship inside the tube lumen of the flexible tube.

15. The ablation catheter system of claim 13, further comprising a cross-shaped fixture located at the distal open end of the catheter, wherein there are four electrical wires and four pre-shaped shape memory splines with one of the four pre-shaped shape memory splines being connected to a respective one of the four electrical wires extending through a respective one of four openings formed by the cross-shaped fixture and the inner surface of the catheter.

16. The ablation catheter system of claim 15, wherein the cross-shaped fixture has a center opening and the flexible tube extends through the center opening in the cross-shaped fixture at the distal open end of the elongated catheter.

17. The ablation catheter system of claim 13, wherein the at least one electrode of the distal electrode array electrically connected to the respective one of the plurality of pre-shaped shape-memory wires has a thermoplastic polyurethane (TPU) cover with a cutout window to expose a surface of the at least one electrode.

18. A catheter assembly, comprising:
a) an elongated catheter comprising an inner surface defining an axial lumen extending from a catheter proximal open end to an opposed distal open end of the catheter;
b) four electrical wires extending from the catheter proximal open end, through the axial lumen and to the distal open end of the catheter;
c) a distal electrode array comprising four pre-shaped shape-memory wires connected to a respective one of the four electrical wires, wherein a plurality of electrodes are electrically connected to each of the four pre-shaped shape-memory wires;
d) a thermoplastic polyurethane (TPU) covering the pre-shaped shape-memory wires, wherein a respective window in the TPU covering exposes each of the plurality of electrodes;
e) a cross-shaped fixture located at the distal open end of the catheter, wherein one of the four pre-shaped shape-memory wires connected to a respective one of the four electrical wires extends through a respective one of four openings formed by the cross-shaped fixture and the inner surface of the catheter;
f) a distal cap connected to a distal end of the four pre-shaped shape-memory wires; and
g) a flexible tube extending through the axial lumen of the catheter and the center opening in the cross-shaped fixture at the distal open end of the elongated catheter, wherein the flexible tube has a tube lumen of a first inner diameter, and a distal end of the flexible tube is spaced proximally with respect to the distal cap; and
h) a guide shaft having a second outer diameter that is less than the first inner diameter of the flexible tube lumen, wherein the guide shaft extends proximally from the distal cap and is received in the tube lumen of the flexible tube, and wherein with the guide shaft received in the tube lumen, an annulus is formed by the tube lumen of the first diameter surrounding the guide shaft of the second diameter,
i) wherein the four pre-shaped shape-memory wires are configured to move between a collapsed position received inside the axial lumen of the catheter, and a basket-shaped, deployed position extending outwardly from the catheter distal open end, and with the four pre-shaped shape-memory wires in the deployed position, the annulus between the outer surface of the guide shaft surrounded by the inner lumen of the flexible tube is sufficient to permit flushing the area adjacent to the deployed splines.

19. The catheter assembly of claim 18, wherein a proximal portion of the guide shaft is in a movable relationship in the tube lumen of the flexible tube.

20. The catheter assembly of claim 18, wherein at least two orientation markers are disposed on a respective one of the four pre-shaped shape-memory wires, and wherein the orientation markers are not electrically connected to the four electrical wires.

21. The catheter assembly of claim 18, further comprising a switching module that is configured to selectively connect a recorder module and an ablation energy generator to the four electrical wires, wherein the recorder module is configured to receive electrical mapping signals from the distal electrode array, and the ablation energy generator is configured to provide electrical ablation energy to the distal electrode array.
